(19) United States
(12) Patent Application Publication
(10) Pub. No.: US 2010/0049114 A1
(43) Pub. Date: Feb. 25, 2010

(54) BLOOD TREATMENT APPLIANCE, STORAGE MEANS FOR USING WITH THE BLOOD TREATMENT APPLIANCE, AND COMBINATION OF THE BLOOD TREATMENT APPLIANCE AND STORAGE MEANS

(76) Inventors: Gail-Suzanne Brown, Usingen-Eschbach (DE); Reiner Spickermann, Wasserlosen-Burghausen (DE)

Correspondence Address: JACOBSON HOLMAN PLLC, 400 SEVENTH STREET N.W., SUITE 600, WASHINGTON, DC, 20004, US

(21) Appl. No.: 12/312,604

(22) PCT Filed: Nov. 19, 2007
(86) PCT No.: PCT/EP2007/009981

§ 371 Date: May 19, 2009

(51) Int. Cl. A61M 1/14; G06F 12/00

(52) U.S. Cl. 604/5.04; 604/4.01; 711/E12.001; 711/115

(57) ABSTRACT

The present invention relates to the field of blood treatment machines such as hemodialysis machines. The object of this invention is to improve upon a generic blood treatment machine such that a certain individualization of the treatment station with little or no expenditure of materials is made possible in order to make the treatment environment more pleasant for the patient to be treated on the machine. The inventive blood treatment machine (10) therefore includes a control unit (16) that is configured so that image files transmitted and/or indexed via a data interface (15) can be displayed on the display screen (13) of the blood treatment machine during a blood treatment. The present invention also includes an external memory means (20) for connection to the data interface (15) and a combination consisting of the blood treatment machine (10) and the external memory means (20).

The invention relates to the field of blood treatment machines according to the preamble of claim 1.

With such blood treatment machines, blood is first withdrawn from a patient for the purpose of treatment of the blood via an extracorporeal blood circulation, then is subjected to a treatment in a blood treatment unit and next is returned to the patient. An example of this is hemodialysis, which is used in kidney replacement therapy. In hemodialysis, the blood treatment unit is a hemodialyzer, which is subdivided by a semipermeable membrane into a blood chamber through which the patient's blood flows and a dialysis fluid chamber through which a cleaning fluid, the dialysis fluid, flows. The semipermeable membrane usually consists of thousands of individual hollow fibers. The substances to be removed from the blood pass by diffusion into the dialysis fluid and are removed with it. At the same time, liquid to be removed by a pressure gradient can be withdrawn from the blood. The blood treatment machine, which is designed as a hemodialysis machine in this case, controls the preparation of the dialysis fluid in the correct composition and temperature and the withdrawal of fluid. At the same time, blood flow in the extracorporeal blood circulation is controlled by controlling a blood pump, usually designed as a roller pump, and monitored for alarm states. Modifications of this form of kidney replacement therapy pertain to hemofiltration or hemodiafiltration in which a fluid exchange is implemented by increased withdrawal of fluid in simultaneous replacement with a substitution solution instead of or in addition to the diffusive purification principle. The blood treatment unit used in this case is also referred to below in general as a hemodialyzer.

In addition to the blood treatment methods mentioned above, there are also other known blood treatment methods with which blood in an extracorporeal circulation is subjected to a treatment. Examples include the use of oxygenators for artificial respiration, adsorber columns for purification of blood or blood centrifuges for separating blood constituents not only for therapeutic purposes but also for donor purposes.

All these forms of treatment have in common the fact that the patient, also understood to refer to donors below, is connected to the blood treatment machine for a long period of time for the blood treatment to be performed. The corresponding period of time may be several hours; in chronic renal replacement therapy it is usually approximately four hours. It is widespread practice for such treatments to be performed in clinics and hospitals where the patients must go regularly—an average of three times a week—for outpatient therapy. Often several patients are treated at the same time in a large room, during which time the patients cannot move any great distance away from the treatment station because of the permanent connection to the blood treatment machine.

These patients usually pass the time during their treatment by watching television, reading or sleeping.

To allow ease of operation of the dialysis machine or at least the determination of the status of the equipment, U.S. Pat. No. 5,903,211 proposes connecting an external entertainment device such as a television set to a hemodialysis machine in such a way that instead of the entertainment program appearing on the television screen, the settings on the hemodialysis machine can be called up and even manipulated there.

However, personalizing the immediate environment of a treatment station is not possible in practical terms because of the boundary hygiene conditions and the constant turnover of patients in a clinic. In particular, personalizing the environment permanently with individual objects such as images is not feasible for this reason and it is also too complicated to constantly be bringing along these items and taking them back again.

The object of the present invention is to improve upon a generic blood treatment machine in such a way as to allow a certain individualization of the treatment station with little or not increased complexity/expense so as to make the treatment environment more pleasant for the patient who must be treated on the machine.

According to the teaching of this invention, this object is achieved by a blood treatment machine having the features of claim 1, an external memory means for use with the inventive blood treatment machine having the features of claim 12 and a combination of a blood treatment machine with an external memory means having the features of claim 14. Advantageous embodiments are the subject matter of the subclaims.

The invention is based on the observation that for individualization of the treatment station, a decisive contribution can be made if it is made possible for the patient to observe images or views selected by the patient during the treatment. Since today's blood treatment machines are generally equipped with enough memory to store control programs and data as well as a display unit designed as a screen, corresponding image files can be displayed on the screen of the blood treatment machine. The image files can be brought along by the patient on a chip card, which is usually available anyway for storing patient data and/or treatment data or on some other suitable memory media such as a USB stick. It may also be sufficient to store the image files on some other memory means that is connected to the control unit of the blood treatment machine and the data brought by the patient may be limited to an indexed selection of images to be displayed. The selection or the image files are transmitted to the blood treatment machine via a data interface connected to the control unit or may be called up by the control unit as needed. The control unit is configured according to this invention so that it displays the image files selected in this way on the screen of the blood treatment machine during a blood treatment. This may take place in a fixed predetermined sequence in the sense of a slide show or it may be predetermined by individual specification by the patient. In the simplest case, just a single image may be displayed.

After the end of the treatment, the display of image files may be terminated automatically so that no retrofitting is required. Instead, an adjustment of the environment can be achieved easily even with the next patient by initiating the next treatment and by transfer of the individual image files or at least the individual image indexing. On the basis of the inventive design of the blood treatment machine, individualization of the treatment station without any significant increase in expense is thus possible.

In one embodiment of the present invention, the blood treatment machine has an internal memory means for storing the image files, said memory means being connected to the control unit for retrieval of the image files.

In another embodiment, the data interface is a USB interface. This widespread type of interface allows a connection of data media such as a USB stick, in particular. Such a USB stick is an example of connection of a data interface to an external memory means. Any other memory means may also be considered here. The memory means may be flexible and mobile in use, such as a USB stick, e.g., in the form of a memory chip card. It is also conceivable to utilize data transmission to a remote memory via a network.

Use of a memory chip card has the advantage that such a card is already in widespread use as a so-called patient card for storing treatment information and/or patient data with the blood treatment machines available on the market. This card may also be used as an external memory means for transmitting or indexing the image files to be displayed in an especially advantageous embodiment of the present invention.

The control unit may also be designed to display the image files as a background image on the display screen. Then blood treatment information displayed on the screen may be partially or completely faded out for display of the image files. In this case, the control unit may be designed so that when predefined conditions occur, the fade is reversed and/or new treatment information is displayed. Such predefined conditions may correspond to alarm states, and the new treatment information may constitute alarm signals. It is also advantageous if the predefined conditions include actuation of the input means. This immediately yields the normal appearance on the display screen for the user when the user operates the input means. The input means may be designed as elements separate from the display screen, e.g., keys or buttons. However, the input means may also include the display screen itself if the screen is designed as a touch-sensitive screen (touchscreen). The invention also includes an external memory means for use with the inventive blood treatment machine, which is designed for connection to the data interface of the blood treatment machine for transmission and/or indexing of the image files to be displayed on the screen during a blood treatment. This external memory means here is preferably a USB stick or a memory chip card, as already explained.

In addition, the present invention includes a combination of the inventive blood treatment machine and the inventive external memory means.

Additional details and advantages of the present invention are described in greater detail below with reference to an exemplary embodiment depicted in the drawings.

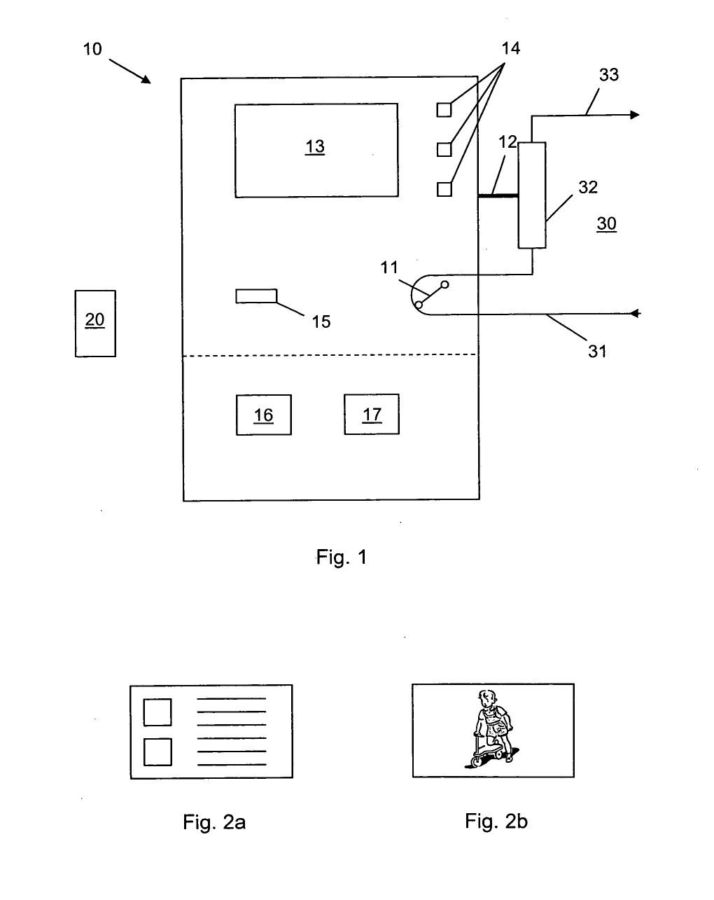

FIG. 1 shows a schematic diagram of an embodiment of the inventive blood treatment machine;

FIG. 2a shows a first view of the screen of the embodiment of the inventive blood treatment machine in which treatment information is displayed; and

FIG. 2b shows a second view of the screen in the embodiment of the inventive blood treatment machine in which an image file is displayed.

FIG. 1 shows an embodiment of an inventive blood treatment machine 10. The blood treatment machine 10 is used for performing a blood treatment with the help of an extracorporeal blood circulation 30, which is not directly part of the blood treatment machine 10, but instead its components are discarded after each treatment. The extracorporeal blood circulation comprises a blood inlet line 31 to supply the blood of a patient to a blood treatment unit 32 and a blood outlet line 33 for returning the blood. The blood treatment machine 10 has means for conveying blood in the extracorporeal circulation in the form of a blood pump 11. FIG. 1 shows only the components that are essential for an understanding of the invention. Those skilled in the art will be aware of the fact that additional components must be added in each case to perform a wide variety of tasks in treating blood.

The blood treatment element 32 is a hemodialyzer in the case when the blood treatment machine 10 is a hemodialysis machine. FIG. 1 shows schematically a holder 12 for holding the blood treatment element 32 on the blood treatment machine 10. No other connections, as in the case of hemodialysis, for supplying and removing the dialysis fluid are shown here.

The blood treatment machine 10 comprises a display screen 13 designed as a touchscreen to simultaneously serve as data entry means. Furthermore, there are also other input means in addition to the screen 13, these additional input means being designed in the form of so-called hard keys 14. Furthermore, a data interface 15, embodied here as a chip card reader or a USB interface, is provided on the front side of the blood treatment machine 10. This also indicates schematically a control unit 16 and an internal memory means 17 which are in the interior of the blood treatment machine 10. The control unit 16, the internal memory means 17, the display screen 13 and the data interface 15 are interconnected via a communications bus (not shown).

The inventive blood treatment machine 10 operates as follows: The patient brings an external memory means designed as a memory chip card 20 along with him to a blood treatment, e.g., a hemodialysis treatment. Patient information such as name, height, weight and information about past treatments such as time, duration and treatment dose as well as the information prescribed by the physician about the treatment to be performed can be stored on the memory chip card 20. In general, the patient does not have direct access to this information, or at least the patient can make only limited changes in it or none at all without special authorization. In addition to this information, however, image files are also stored on the memory chip card 20; these files were already loaded previously by the patient onto the memory chip card 20 in a memory area reserved for this purpose.

The memory chip card, designed as a read-write device, is then inserted into the data interface 15 prior to the treatment. The control unit 16 is configured so that it can read the treatment information for the treatment to be performed from the memory chip card 20 and store it in the internal memory means 17. The image files which are preferably stored on the chip card in a conventional compressed format such as JPEG, TIFF or PDF are likewise transferred to the internal memory means 17. In addition, information about the order in which the image files are to be displayed later on the display screen 13 may also be stored on the memory chip card 20. It is also possible to provide for a type of directory of available image files to be displayed for the patient on the display screen 13 and the patient can then select the sequences to be displayed with the help of the touch-sensitive display screen 13 and/or the hard keys 14. For those skilled in the art, a wide variety of selection techniques will be available for this purpose. A memory area can be reserved for each patient in the internal memory means 17 so that the image files can also be available for subsequent treatments. Then it may be sufficient to simply store the image sequences in an indexed form on the memory chip card 20 to be able to display the image files already stored in the internal memory means 17 during a treatment.

In the embodiment of the data interface 15 as a USB interface, it is self-evident to have the images stored on the external memory means, which may be in the form of a USB stick having an enormous memory capacity in this case. In principle, the image files may also be in the form of film and movie files if sufficient memory capacity is available, in which case the terminology “image file” as used within the context of the present patent application shall also be understood to explicitly include film and movie files.

After connecting the patient to the lines of the extracorporeal blood circulation 30 and starting the blood treatment, the control unit 16 begins to fade in the screen view previously used for display of the treatment information (treatment information mode) as indicated highly schematically in FIG. 2a. Instead, the image files selected and played by the patient are faded in (image display mode), as indicated schematically in FIG. 2b. Some important treatment information may also be faded in (not shown), so the images then appear as a background image.

If the control unit 16 ascertains that, because of the occurrence of preselected conditions such as alarm states, touching of the display screen 13 or the operation of the hard keys 14 should be returned for the purpose of displaying treatment information, then the control unit will alter the screen display accordingly. In particular, in the case of alarm states, other optical and/or acoustic signals will also be output at the same time, if necessary, to draw the attention of the patient or the treatment personnel to the alarm state.

By operating a certain input means, e.g., a certain hard key 14, the user may select the return to the image display mode. Under certain circumstances, the control unit 16 may also automatically change to this display mode after a predetermined period of time has elapsed. It is also possible to provide for the image display mode to be activated by the control unit 16, in general, as a type of screensaver after a predetermined period of time.

It is also possible to provide for the control unit 16 to switch immediately to the treatment information mode whenever there is an interruption in data interface 15, e.g., after the external memory means 20 is removed, and optionally to then block access to the image files stored in the internal memory means 17 for that patient.

With the help of the inventive blood treatment machine, it is possible to individualize the treatment station for a patient who must receive a blood treatment as an outpatient for several hours and thus to ensure increased feelings of well-being in the patient during the blood treatments. By playback of images with which the patient is familiar, e.g., by displaying images of relatives or landscapes, it is possible to increase the value of the treatment environment, which otherwise tends to be not very individual.

1. A blood treatment machine (10) for treating the blood of a patient, wherein blood is sent from a patient to a blood treatment unit (32) and from this unit back to the patient, comprising
a display screen (13) for display of information pertaining to the blood treatment,
input means (13, 14),
an electronic data interface (15) and
a control unit (16) connected to the electronic data interface (15) and to the display screen (13),
characterized in that
the control unit (16) is configured so that image files transmitted and/or indexed via the data interface (15) are displayed on the display screen (13) during a blood treatment.

2. The blood treatment machine according to claim 1,
characterized in that
the blood treatment unit (32) is a hemodialyzer.

3. The blood treatment machine according to claim 1,
characterized in that
it has an internal memory means (17) for storing the image files, said means being connected to the control unit (16).

4. The blood treatment machine according to claim 1,
characterized in that
the data interface (15) is designed for connection to an external memory means (20) outside of the blood treatment machine.

5. The blood treatment machine according to claim 4,
characterized in that
the data interface (15) is a USB interface.

6. The blood treatment machine according to claim 4,
characterized in that
the external memory means (20) is a memory chip card or a USB memory stick.

7. The blood treatment machine according to claim 1,
characterized in that
the control unit (16) is designed so that it displays the image files as background image on the display screen (13).

8. The blood treatment machine according to claim 1,
characterized in that
the control unit (16) is designed so that it partially or completely fades out blood treatment information being displayed, so that the image files can be displayed.

9. The blood treatment machine according to claim 8,
characterized in that
the control unit (16) is also designed so that it reverses the fadeout when predefined conditions occur and/or displays new treatment information.

10. The blood treatment machine according to claim 9,
characterized in that
the predefined conditions correspond to alarm states and the new treatment information constitutes alarm signals.

11. The blood treatment machine according to claim 8,
characterized in that
the predefined conditions comprise actuation of the input means (13, 14).

12. The external memory means (20) for use with a blood treatment machine (10) according to claim 1,
characterized in that
it is designed for connection to the data interface (15) of the blood treatment machine (10) for transmission and/or indexing of the image files to be displayed on the display screen (13) of the blood treatment machine (10) during a blood treatment.

13. The external memory means according to claim 12,
characterized in that
it is a USB stick or a memory chip card.

14. A combination comprising a blood treatment machine according to claim 1 and an external memory medium configured for connection to the data interface of the blood treatment machine for at least one of transmitting and indexing of the image files to be displayed on the display screen of the blood treatment machine during the blood treatment.

15. The combination according to claim 14, wherein the external memory medium is a USB stick or a memory chip card.
